(19) United States
(12) Plant Patent Application Publication
(10) Pub. No.: US 2004/0148672 P1
(43) Pub. Date: Jul. 29, 2004

(54) Rhaphiolepsis plant named 'Corleyscourii'

(75) Inventor: Willie Lee Corley, Griffin, GA (US)

Correspondence Address: One World Trade Center, Suite 1600, 121 S.W. Salmon Street, Portland, OR, 97204, US

(73) Assignee: Athena Trees, Inc.

(21) Appl. No.: 10/352,337

(22) Filed: Jan. 27, 2003

(51) Int. Cl. A01H 5/00

(52) U.S. Cl. PLT/254

(57) ABSTRACT

A Rhaphiolepsis×delacouri’ plant named ‘Corleyscourii’ characterized by its resistance to leaf spot, burgundy winter foliage, compact habit, cold hardiness and uniform floriferousness.

### LATIN NAME OF THE GENUS AND SPECIES OF THE PLANT CLAIMED

Rhaphiolepis×delacourii

### VARIETY DENOMINATION

‘Corleyscourii’

### BACKGROUND

Rhaphiolepis×delacourii ‘Corleyscourii’ is the result of a cross between R. umbellata and R. indica. Rhaphiolepis umbellata is a shrub native to Japan and Korea while R. indica hails from China. Rhaphiolepis umbellata and R. indica where first crossed by one Delacour (hence ×delacourii), in Cannes, France, in the late nineteenth century. Rhaphiolepis species are grown chiefly as ornamental flowering and evergreen shrubs for low hedges, groupings, and mass plantings.

The present invention relates to a new and distinct variety of Rhaphiolepis×delacourii which is characterized by its resistance to leaf spot disease caused by Entomosporium spp., seasonal foliage color, compact habit, cold hardiness, and uniform floriferousness. This variety is the result of efforts to identify and propagate Indian Hawthorn varieties which are aesthetically pleasing and resistant to pathogens which have disfigured plantings of Rhaphiolepis throughout the southeastern U.S., in particular leaf spot, Entomosporium spp.

All progeny of Rhaphiolepis×delacourii ‘Corleyscourii’ are derived from a single disease-resistant individual original plant which was grown from an open-pollinated seed which was collected from a single Rhaphiolepis×delacourii ‘Georgia Petite’ (U.S. Plant Pat. No. 9,983) growing in Griffin, Ga. Thus ‘Corleyscourii’ has ‘Georgia Petite’ as its seed parent and is similar to ‘Georgia Petite’ in its compact habit, disease resistance, and cold hardiness. The new variety differs from ‘Georgia Petite’ in that the dark green evergreen foliage of the new variety provides exceptional winter interest by turning a beautiful rich burgundy.

### BRIEF SUMMARY

Rhaphiolepis×delacourii ‘Corleyscourii’ has been asexually propagated at a location near Athens, Ga. by softwood cuttings. The observations below, unless otherwise indicated, are from asexually propagated progeny growing in Monroe, Ga. Observations of my new variety have confirmed that the following unique combination of characteristics remain fixed in succeeding generations and distinguish Rhaphiolepis×delacourii ‘Corleyscourii’.

1. Leaf spot- and fire blight-resistant foliage (resistant to Entomosporium spp.).

2. Evergreen leaves that offer highly ornamental, uniform, burgundy winter leaf coloration. The richness of the burgundy color is a desirable feature in the winter landscape.

3. Cold hardiness and heat tolerance.

4. Growth that has proven to be exceptionally tight and compact, with dense foliage.

5. Uniform flowering throughout the expanse of the plant. ‘Corleyscourii’ has not been observed to become leggy and tight branching occurs even at the base of the plant, thus creating uniform flowering over the entire canopy.

### BRIEF DESCRIPTION OF THE DRAWINGS

The accompanying photographs depict the color of the plant, flowers and foliage of my new variety as nearly as is reasonably possible to make the same in a color illustration of this character. It should be noted that colors in a photograph can vary with lighting conditions. Also, growing conditions can affect plant color.

FIG. 1. The new variety in its winter leaf coloration: The dark green of the summer leaves has become an attractive rich dark burgundy.

FIG. 2. The new variety when flowering. The plant is a small rounded shrub covered in white flowers. The mound exhibits tight branching from the base to the top (no legginess), and displays a mantle of flowers from the ground up.

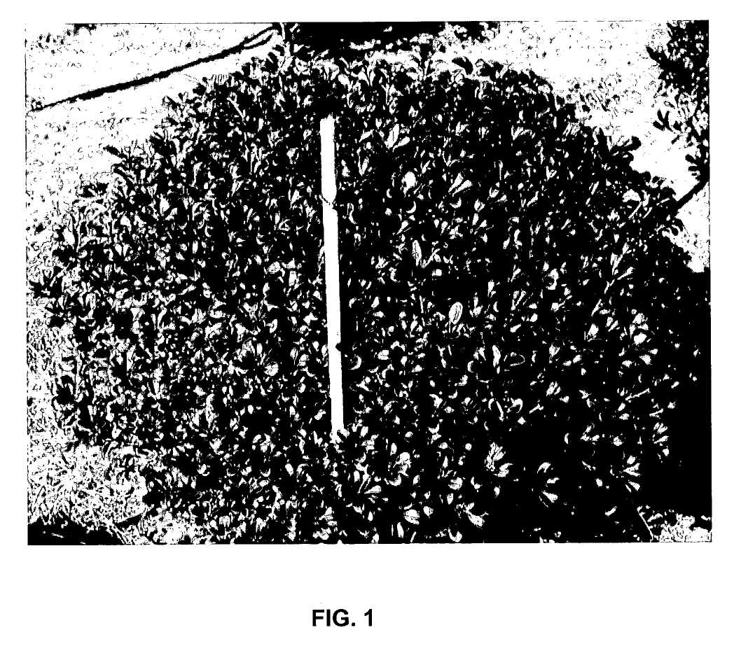

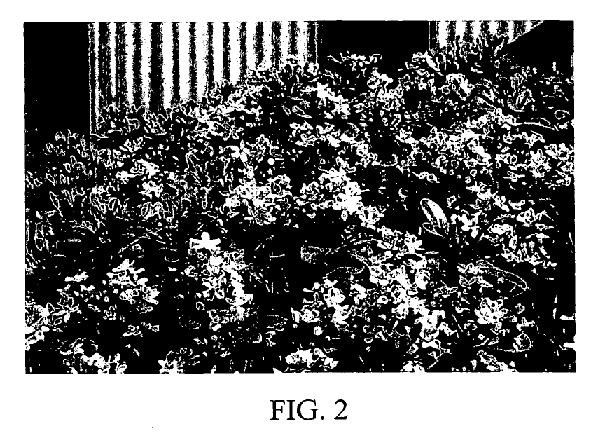

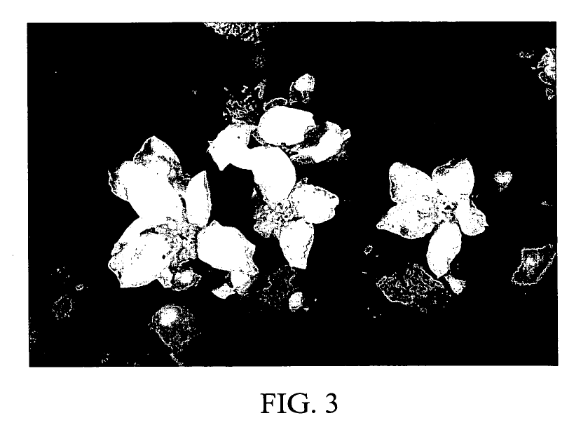

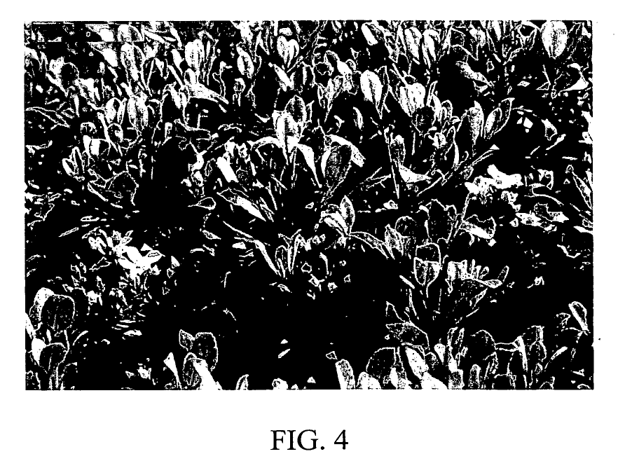

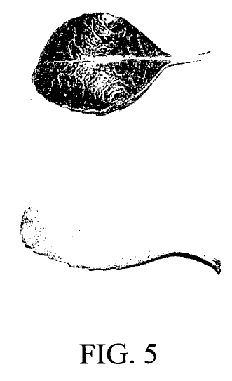

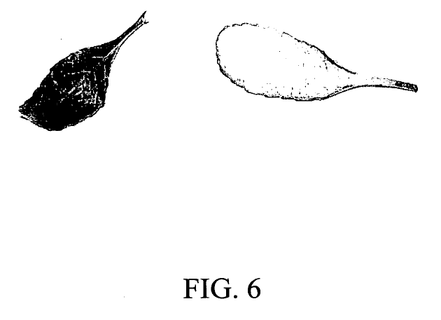

### DETAILED BOTANICAL DESCRIPTION

The following is a botanical description of Rhaphiolepis×delacourii ‘Corleyscourii’ based on a specimen grown in the field at Monroe, Ga. with color references pursuant to The Royal Horticultural Society Colour Chart (RHS).

Scientific name: Rhaphiolepis×delacourii, Varietal Name ‘Corleyscourii’.

Parentage:

Seed parent.—Rhaphiolepis×delacourii ‘Georgia Petite”.

Pollen parent.—Unknown.

Propagation: ‘Corleyscourii’ is easily reproduced, true to type, from softwood cuttings. Tip cuttings are taken when the first flush of growth hardens (June in Monroe, Ga.). Cuttings are dipped for 3 to 5 seconds in 5000 ppm KIBA. Cuttings are stuck under mist in a lightweight media and usually root at a rate of 75% or better in 6 to 8 weeks.

Habit:

Habit.—Low, rounded, tight, and dense, with multiple shoots arising in the spring from vegetative buds formed the previous season around the terminus of the branch. Average number of breaks is 10.7 (range from 6 to 18 new shoots). Internodes are short, averaging 1 cm, resulting in the tight form.

Rate.—Vigorous when young, but slows with age resulting in an average growth rate of 20 cm per year. The observed plant was only 66 cm high and 102 cm wide after 4 years.

Leaves: Evergreen, simple, arranged alternately, internodes are short (averaging 1 cm) with leaves mostly near the end of a season's growth (could almost appear whorled).

Shape.—obovate to elliptic.

Margin.—Serrate, undulate, those on the outside of canopy with a burgundy margin in summer (RHS greyed-purple group 187A).

Base.—Cuneate.

Apex.—Obtuse.

Surface.—Leathery, thick, glabrous. at maturity, 7 to 8 vein pairs.

Length and width.—5.72 cm long and 2.54 cm wide.

Color.—Summer: Upper surface: Flat dark green (RHS yellow-green group 147A). Underside: Flat pale green (RHS yellow-green group 147D). Margin: Burgundy (RHS greyed-purple group 187A) for those on outside of canopy. Winter: Burgundy (RHS greyed-purple group 187A). New leaves: Medium green (RHS yellow-green group 146C) with beige (RHS greyed-orange group 165C) tomentum.

Petiole.—1.27 cm long, flat, light green (RHS yellow-green group 145B), glabrous.

Buds.—Terminal buds: produce flowers and vegetative growth in the spring from buds that are solitary, conical, scales imbricate, 3 scales, 1/16 inches high and wide, light green (RHS yellow-green group 145B) and produce vegetative growth again in mid-summer from buds that are solitary, conical, scales imbricate, 1/16 inches high and wide, light brown (RHS grey-brown group 199D) with white (RHS white group 155D) pubescence. Lateral buds: ensconced in the bark; buds observed as of this time to only break into growth when terminal is pruned.

Stems:

Surface.—1st year—bright green (RHS yellow-green group 144C) with beige (RHS greyed-orange group 165C) tomentum; 2nd year—flat light brown (RHS grey-brown group 199D) with vertical fissures.

Pith.—solid, uniform, pale green (RHS green group 139D) with pale yellow (RHS yellow-green group 150D) vascular tissue.

Leaf scar.—2 mm wide, 1 mm high, crescent-shaped, 3 bundle traces.

Flowers:

Color.—White (RHS white group 155D) with rose (RHS red-purple group 65D) markings.

Size.—1.91 cm diameter flowers; each petal 9.53 mm high, 6.35 mm wide, entire.

Botanical description.—Rosaceous flower with 5 petals, 5 sepals, 15 stamens, 3 styles to 1 ovary. Stamens with white (RHS white group 155D) filament and yellow (RHS yellow group 3D) anthers. After anthesis filament turns pink (RHS red-purple group 62C) then red (RHS red-purple group 59A) and anthers brown (RHS greyed-orange group 165A) and a brown (greyed-orange group 164B) pubescence develops at base of petals.

Inflorescence type.—Panicle, 5.08 to 7.62 cm wide, 5.08 to 7.62 cm high, average of 16 flowers per inflorescence.

Season.—May, in Monroe, Ga., Zone 7b.

Fragrance.—none observed.

Fruit:

Type.—berry.

Color.—Green turning flat dark purple (RHS greyed-purple group 187A) ageing to black (RHS group 202A).

Size.—Round, 6.35 mm diameter.

Season.—Mature in September and persist into March (when they dry up), in Monroe, Ga., Zone 7b.

Disease resistance: After 12 years in field growing conditions surrounded with plants infected with leaf spot, Entomosporium spp., ‘Corleyscourii’ remained vigorous and disease free while less resistant varieties were disfigured by the unsightly leaf spot disease. In separate evaluations in nurseries in Athens, Ga., Cairo, Ga., and Houston, Tex., ‘Corleyscourii’ remained exceptionally free of disease, including leaf spot and fire blight, in greenhouse settings and outside in propagation areas under overhead irrigation.

I claim:

1. A new and distinct variety of Raphiolepsis×delacourii, substantially as herein shown and described.
